(19) United States
(12) Patent Application Publication
(10) Pub. No.: US 2003/0176327 A1
(43) Pub. Date: Sep. 18, 2003

(54) Antibiotics for treating biohazardous bacterial agents

(76) Inventors: Gail Houston Cassell, Carmel, IN (US); Thalia Ioanna Nicas, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/274,692

(22) Filed: Oct. 18, 2002

Related U.S. Application Data
(60) Provisional application No. 60/335,304, filed on Oct. 31, 2001.
(60) Provisional application No. 60/346,008, filed on Oct. 25, 2001.

(51) Int. Cl. A61K 38/16

(52) U.S. Cl. 514/8

(57) ABSTRACT

The present invention is directed to methods for the control of strains of bio-hazardous bacterial agents. These agents include: Bacillus anthracis, Yersinia Pestis, Francisella tularensis, Clostridium botulinin, Clostridium Perfringens, Brucella abortis, B milletensis, B suis and Burkholderia mallei. These methods employ treating an infected warm-blooded animal with an antibiotics selected from: Cephalothin, Cefazolin, Cephalexin monohydrate, Cephalexin HCl, Cefaclor, Loracarbef, Erythromycin estolate, Dirithromycin, Cinoxacin, Vancomycin HCl, Tobramycin, Cefamandole, Cefuroxime, Daptomycin, and Oritavancin.

### BACKGROUND OF THE INVENTION

Of the numerous bio-hazardous bacterial agents that may be used as weapons, there are a limited number of organisms that could cause disease and deaths in sufficient numbers to cripple a city or region. These organisms include: Bacillus anthracis, Yersinia Pestis, Francisella tularensis, Clostridium botulinin, Clostridium, Perfringens, Brucella abortis, B milletensis, B suis and Burkholderia mallei. Anthrax, attributable to infection with Bacillus anthracis, is among the most serious diseases that can be contracted from a bio-hazardous agent.

High hopes were once vested in the Biological Weapons and Toxins Convention, which prohibited offensive biological weapons research or production and was signed by most countries. However, Iraq and the former Soviet Union, both signatories of the convention, have subsequently acknowledged having offensive biowarfare programs; a number of other countries are believed to have such programs, as have some autonomous terrorist groups. The possibility of a terrorist attack using bioweapons would be especially difficult to predict, detect, or prevent, and thus, it is among the most feared terrorist scenarios (see A. Carter, J. Deutsch, and P. Zelicow, “Catastrophic terrorism,” Foreign Aff. 77, 80-95 (1998))

Biological agents have seldom been dispersed in aerosol form, the exposure mode most likely to inflict widespread disease. Therefore, historical experience provides little information about the potential impact of a biological attack or the possible efficacy of postattack measures such as vaccination, antibiotic therapy, or quarantine.

For centuries, anthrax has caused disease in animals and, uncommonly, serious illness in humans throughout the world. (see D. Lew , Bacillus anthracis (anthrax); in G. L. Mandell, J. E. Bennett, R. Dolin, eds.; Principles and Practices of Infectious Disease, New York, N.Y.; Churchill Livingstone Inc; 1885-1889 (1989)). Research on anthrax as a biological weapon began more than 80 years ago. (see G. W. Christopher, T. J. Cieslak, J. A. Pavlin, and E. M. Eitzen, “Biological warfare: a historical perspective,” JAMA 278, 412-417 (1997)). Today, at least 17 nations are believed to have offensive biological weapons programs (see L. A. Cole, “The specter of biological weapons,” Sci.Am., 60-65 (December 1996)); it is uncertain how many are working with anthrax. Iraq has acknowledged producing and weaponizing anthrax. (see R. A. Zalinskas, “Iraq's biological weapons: the past as future?,” JAMA 278, 418-424 (1997)).

Most experts concur that the manufacture of a lethal anthrax aerosol is beyond the capacity of individuals or groups without access to advanced biotechnology. However, autonomous groups with substantial funding and contacts may be able to acquire the required materials for a successful attack. One terrorist group, Aum Shinrikyo, responsible for the release of sarin in a Tokyo, Japan, subway station in 1995, (see Public Health Service Office of Emergency Preparedness, “Proceedings of the Seminar on Responding to the Consequences of Chemical and Biological Terrorism,” Washington, D.C.: US Dept. of Health and Human Services (1995), dispersed aerosols of anthrax and botulism throughout Tokyo on at least 8 occasions. For unclear reasons, the attacks failed to produce illness. (see S. WuDunn, J. Miller, and W. Broad, “How Japan germ terror alerted world,” New York Times, 1-6 (May, 1998)).

The accidental aerosolized release of anthrax spores from a military microbiology facility in Sverdlovsk in the former Soviet Union in 1979 resulted in at least 79 cases of anthrax infection and 68 deaths and demonstrated the lethal potential of anthrax aerosols. (see M. Meselson, J. Guillemin, M. Hugh-Jones, et al., “The Sverdlovsk anthrax outbreak of 1979,” Science 266, 1202-1208 (1994)). An anthrax aerosol would be odorless and invisible following release and would have the potential to travel many kilometers before disseminating. (see World Health Organization, “Health Aspects of Chemical and Biological Weapons”, Geneva, Switzerland: World Health Organization, 98-99 (1970) and J. D. Simon, “Biological terrorism: preparing to meet the threat,” JAMA 278, 428-430 (1997)). Evidence suggests that following an outdoor aerosol release, persons indoors could be exposed to a similar threat as those outdoors. (see G. A. Christy and C. V. Chester, “Emergency Protection Against Aerosols,” Oak Ridge, Tenn: Oak Ridge National Laboratory (1981), Publication ORNL-5519).

In 1970, a World Health Organization (WHO) expert committee estimated that casualties following the theoretical aircraft release of 50 kg of anthrax over a developed urban population of 5 million would be 250,000, 100,000 of whom would be expected to die without treatment.⁹ A 1993 report by the US Congressional Office of Technology Assessment estimated that between 130,000 and 3 million deaths could follow the aerosolized release of 100 kg of anthrax spores upwind of the Washington, D.C., area-lethality matching or exceeding that of a hydrogen bomb. (see Office of Technology Assessment, U.S. Congress, “Proliferation of Weapons of Mass Destruction,” Washington, D.C.: US Government Printing Office; 53-55 (1993), Publication OTA- ISC-559. An economic model developed by the Centers for Disease Control and Prevention (CDC) suggested a cost of $26.2 billion per 100,000 persons exposed. (see A. F. Kaufmann, M. I. Meltzer and G. P. Schmid, “The economic impact of a bioterrorist attack,” Emerg Infect Dis. 3, 83-94 (1997)).

There are no clinical studies of the treatment of inhalational anthrax in humans. Thus, antibiotic regimens commonly recommended for empirical treatment of sepsis have not been studied in this setting. In fact, natural strains of B anthracis are resistant to many of the antibiotics used in these empirical regimens, such as those of the extended-spectrum cephalosporins. (see M. W. Odendaal, P. M. Peterson, V. de Vos, and A. D. Botha, “The antibiotic sensitivity patterns of Bacillus anthracis isolated from the Kruger National Park, Onderstepoort J Vet Res. 58, 17-19 (1991) and M. Doganay and N. Aydin, “Antimicrobial susceptibility of Bacillus anthracis,” Scand J Infect Dis. 23, 333-335 (1991)). Most naturally occurring anthrax strains are sensitive to penicillin, and penicillin historically has been the preferred therapy for the treatment of anthrax. Penicillin is approved by the FDA for this indication, (see D. R. Franz, P. B. Jahrling, A. Friedlander, et al., “Clinical recognition and management of patients exposed to biological warfare agents,” JAMA 278, 399-411 (1997), J. M. Barnes, “Penicillin and B anthracis,” J Pathol Bacteriol 194, 113-125 (1947) and R. E. Lincoln, F. Klein, J. S. Walker, et al., “Successful treatment of monkeys for septicemic anthrax,” In: Antimicrobial Agents and Chemotherapy -1964, Washington, D.C.: American Society for Microbiology, 759-763 (1965) as is doxycycline. (see American Hospital Formulary Service, AHFS Drug Information, Bethesda, Md: American Society of Health System Pharmacists (1996)). In studies of small numbers of monkeys infected with susceptible strains of B anthracis, oral doxycycline has proved efficacious (see D. R. Franz, P. B. Jahrling, A. Friedlander, et al., “Clinical recognition and management of patients exposed to biological warfare agents,” JAMA 278, 399-411 (1997)).

Doxycycline is the preferred option from the tetracycline class of antibiotics because of its proven efficacy in monkey studies and its ease of administration. Other members of this class of antibiotics are suitable alternatives. Although treatment of anthrax infection with ciprofloxacin has not been studied in humans, animal models suggest excellent efficacy. (see A. Friedlander, S. L. Welkos, M. L. Pitt, et al., “Postexposure prophylaxis against experimental inhalation anthrax,” J Infect Dis 167, 1239-1242 (1993); D. R. Franz, P. B. Jahrling, A. Friedlander, et al., “Clinical recognition and management of patients exposed to biological warfare agents,” JAMA 278, 399-411 (1997) and D. Kelly, J. D. Chulay, P. Mikesell and A. Friedlander, “Serum concentrations of penicillin, docycycline, and ciprofloxacin during prolonged therapy in rhesus monkeys,” J Infect Dis 166, 1184-1187 (1992)) In vitro data suggest that other fluoroquinolone antibiotics would have equivalent efficacy in treating anthrax infection, although no animal data exist for fluoroquinolones other than ciprofloxacin. (see M. Doganay and N. Aydin, “Antimicrobial susceptibility of Bacillus anthracis,” Scand J Infect Dis. 23, 333-335 (1991))

Reports have been published of a B anthracis vaccine strain that has been engineered by Russian scientists to resist the tetracycline and penicillin classes of antibiotics. (see A. V. Stepanov, L. I. Marinin, A. P. Pomerantsev and N. A. Staritsin, “Development of novel vaccines against anthrax in man,” J Biotechnol 44, 155-160 (1996) Although the engineering of quinolone-resistant B anthracis may also be possible, to date there have been no published accounts of this.

Other antibiotics effective against B anthracis in vitro include chloramphenicol, erythromycin, clindamycin, extended-spectrum penicillins, macrolides, aminoglycosides, vancomycin hydrochloride, cefazolin, and other first-generation cephalosporins. (see M. W. Odendaal, P. M. Peterson, V. de Vos, and A. D. Botha, “The antibiotic sensitivity patterns of Bacillus anthracis isolated from the Kruger National Park, Onderstepoort J Vet Res. 58, 17-19 (1991); M. Doganay and N. Aydin, “Antimicrobial susceptibility of Bacillus anthracis,” Scand J Infect Dis. 23, 333-335 (1991)) and N. F. Lightfoot, R. J. Scott and P.C. Turnbull, “Antimicrobial susceptibility of Bacillus anthracis: proceedings of the international workshop on anthrax,” Salisbury Med Bull. 68, 95-98 (1990)). The efficacy of these antibiotics has not been tested in humans or animal studies. The working group recommends the use of these antibiotics only if the previously cited antibiotics are unavailable or if the strain is otherwise antibiotic resistant. Natural resistance of B anthracis strains exists against sulfamethoxazole, trimethoprim, cefuroxime, cefotaxime sodium, aztreonam, and ceftazidime. (see M. W. Odendaal, P. M. Peterson, V. de Vos, and A. D. Botha, “The antibiotic sensitivity patterns of Bacillus anthracis isolated from the Kruger National Park, Onderstepoort J Vet Res. 58, 17-19 (1991); M. Doganay and N. Aydin, “Antimicrobial susceptibility of Bacillus anthracis,” Scand J Infect Dis. 23, 333-335 (1991)) and N. F. Lightfoot, R. J. Scott and P. C. Turnbull, “Antimicrobial susceptibility of Bacillus anthracis: proceedings of the international workshop on anthrax,” Salisbury Med Bull. 68, 95-98 (1990)) Therefore, these antibiotics should not be used in the treatment or prophylaxis of anthrax infection.

There is a need for still other antibiotics for treating infections caused by biohazardous agents.

### BRIEF SUMMARY OF THE INVENTION

The present invention is directed to methods for the control of bio-hazardous bacterial agents. Exemplary agents include: Bacillus anthracis, Yersinia Pestis, Francisella tularensis, Clostridium botulinin, Clostridium Perfringens, Brucella abortis, B milletensis, B suis and Burkholderia mallei. These methods employ treating an infected warm-blooded animal with an antibiotics selected selected from: Cephalothin, Cefazolin, Cephalexin monohydrate, Cephalexin HCl, Cefaclor, Loracarbef, Erythromycin estolate, Dirithromycin, Cinoxacin, Vancomycin HCl, Tobramycin, Cefamandole, Cefuroxime, Daptomycin, and Oritavancin.

### DETAILED DESCRIPTION OF THE INVENTION

As used herein:

The term “Bio-hazardous bacterial agent” means a bacterial organism that may be used as a weapon to cause disease and deaths in sufficient numbers to cripple a city or region. Exemplary bio-hazardous bacterial agents include: Bacillus anthracis, Yersinia Pestis, Francisella tularensis, Clostridium botulinin, Clostridium, Perfringens, Brucella abortis, B milletensis, B suis and Burkholderia mallei. A particularly hazardous agent is Bacillus anthracis.

The terms “Cephalothin,” “Cefazolin,” “Cephalexin monohydrate,” “Cephalexin HCl,” “Cefaclor,” “Loracarbef,” “Erythromycin estolate,” “Dirithromycin,” “Cinoxacin,” “Vancomycin HCl,” “Tobramycin,” “Cefamandole,” and “Cefuroxime” refer to commercial antibiotics in their usual usage of the term, as understood by one of ordinary skill in the art. For example, “Cefaclor” is a semi-synthetic cephalosporin antibiotic for oral administration. It is chemically designated as 3-chloro-7-D-(2-phenylglycinamido)-3-cephem-4-carboxylic acid monohydrate. “Loracarbef” is a synthetic β-lactam antibiotic of the carbacephem class for oral administration. Chemically, carbacephems differ from cephalosporin-class antibiotics in the dihydrothiazine ring where a methylene group has been substituted with a sulfur atom. The chemical name for loracarbef is (6R,7S)-7-[(R)-2-amino-2-phenylacetamido]-3-chloro-8-oxo-1-azabicyclo[4.2.0]oct-2-ene-2-carboxylic acid, monohydrate. “Vancomycin” is a natural product glycopeptide antibiotic for oral or intravenous administration, preferably intravenous administraton. Finally, “Cefalexin” is a semi-synthetic cephalosporin antibiotic for oral administration.

The term “Daptomycin” means N-(1-oxodecyl)-L-tryptophyl-L-asparaginyl-L-A-aspartyl-L-threonylglycyl-L-ornithyl-L-A-aspartyl-D-alanyl-L-A-aspartylglycyl-D-seryl-threo-3-methyl-L-A-glutamyl-G-(2-aminophenyl)-G-oxo-L-A-aminobutanoicacid, E1-lactone. Daptomycin is described in U.S. Pat. No. 4,537,717, which is incorporated herein by reference (see Compound 4). Daptomycin can be administered orally or intravenously.

The term “Oritavancin” means N^{DISACC}-(4-(4-chlorophenyl)benzyl)A82846B, or a salt thereof. Oritavancin is a semi-synthetic antibiotic of the glycopeptide class. N^{DISACC}-(4-(4-chlorophenyl)benzyl)A82846B is (4″R)-22-O-(3-amino-2,3,6-trideoxy-3-C-methyl-A-L-arabino-hexopyranosyl)-N3″-((4-chloro(1,1′-biphenyl)-4′-yl)methyl)vancomycin. N^{DISACC}-(4-(4-chlorophenyl)benzyl)A82846B and salts thereof are described in U.S. Pat. No. 5,840,684, which is incorporated herein by reference (see Example 229). N^{DISACC}-(4-(4-chlorophenyl)benzyl)A82846B can be administered orally or intravenously, preferably intravenously.

In the present invention, an antibacterially effective amount of a compound selected from the group consisting of Cephalothin, Cefazolin, Cephalexin monohydrate, Cephalexin HCl, Cefaclor, Loracarbef, Erythromycin estolate, Dirithromycin, Cinoxacin, Vancomycin HCl, Tobramycin, Cefamandole, Cefuroxime, Daptomycin, and Oritavancin is used to treat a host, warm-blooded animal suffering a bacterial infection attributable to a bio-hazardous bacterial agent. Preferably, the compound is cephalexin monohydrate, cefaclor, or cefacandole. The suitabilities of individual antibiotics for treating particular strains of bio-hazardous bacterial agents are shown in Tables 1 and 2.

The present invention is practiced in the usual mode of antibacterial therapy. The subject compound or a salt thereof is administered to the host animal. The compound can sometimes be successfully administered on a single occasion, but is more commonly administered at intervals over a period of days to assure control. Administration can be by the oral route or by a parenteral route.

The exact dose to be employed is not critical, and will vary with the host, the particular strain, and other factors known to the clinician. The dose must be high enough to ensure adequate concentration of the compound in the host's tissues. Dosing regimens for Cephalexin HCl, Cefaclor, Loracarbef, Erythromycin estolate, Dirithromycin, Cinoxacin, Vancomycin HCl, Tobramycin, Cefamandole, and Cefuroxime are well known in the art and are described, for example, on the product package inserts.

When the active ingredient is daptomycin, an effective dose is generally between about 0.1 and about 100 mg/kg. A preferred dose is from about 1 to about 30 mg/kg. A typical daily dose for an adult human is from aout 100 mg to about 1.0 g.

When the active ingredient is oritavancin, doses of from about 1 mg/kg to about 25 mg/kg are efficacious in the present invention; preferred doses are from about 1.5 mg/kg/day to about 5 mg/kg/day. A typical daily dose for an adult human is from about 100 mg to about 500 mg.

In the normal practice of pharmaceuticals, the active compound to be employed in the present invention is preferably formulated with one or more adjuvants, carriers, and/or diluents. The identity of suitable such components is well known to those skilled in the art, as is the method of mixing such components. For oral administration, the subject compound can be formulated as a capsule, tablet, suspension, or other form for oral delivery. For intravenous infusion, the compound can be dissolved in a suitable intravenous fluid, such as physiological saline, 5% dextrose solution, or the like.

In vitro methods for testing the biological activity of antibiotics against Bacillus anthracis are well known to those of ordinary skill in the art (see, e.g., M. Doganay and N. Aydin, “Antimicrobial susceptibility of Bacillus anthracis.” Scand J Infect Dis. 23, 333-335 (1991); N. F. Lightfoot, R. J. Scott, and P. C. Turnbull, “Antimicrobial susceptibility of Bacillus anthracis: proceedings of the international workshop on anthrax,” Salisbury Med Bull. 68, 95-98 (1990); and M. W. Odendaal, P. M. Peterson, V. de Vos, and A. D. Botha, “The antibiotic sensitivity patterns of Bacillus anthracis isolated from the Kruger National Park,” Onderstepoort J Vet Res. 58, 17-19 (1991)).

TABLE I
Suitability of selected antibiotics against
specific biohazard bacterial agents

| | Bacillus | Yersinia | Francisella |
|---|---|---|---|
| | anthracis | Pestis | tularensis |
| Oral Cephalosporins | Yes | Yes | Yes |
| and Carbacephalsporins | | | |
| Cephalothin | Yes | Yes | Yes |
| Cefazolin | Yes | Yes | Yes |
| Cephalexin | Yes | Yes | Yes |
| Monohydrate | | | |
| Cefaclor | Yes | Yes | Yes |
| Loracarbef | Yes | Yes | Yes |
| Other Classes of Oral | Yes | Yes | Yes |
| Agents | | | |
| Erythromycin Estolate | Yes | Yes | Yes |
| Dirithromycin | Yes | Yes | Yes |
| Cinoxacin | Yes | Yes | Yes |
| Injectable agents | Yes | Yes | Yes |
| Vancomycin HCl | Yes | No | No |
| Tobramycin | Yes | Yes | Yes |
| Cefamandole | Yes | Yes | Yes |
| Cefuroxime | Yes | Yes | Yes |
| Daptomycin | Yes | No | No |
| Oritavancin | Yes | No | No |

TABLE II
Suitability of selected antibiotics against
specific biohazard bacterial agents

| | | | Brucella abortis, | |
|---|---|---|---|---|
| | Clostridium | Clostridium | B milletensis and | Burkholderia |
| | botulinin | perfringens | B suis | mallei |
| Oral | No | No | No | No |
| Cephalosporins | | | | |
| and | | | | |
| Carbacephalsporins | | | | |
| Cephalothin | No | No | No | No |
| Cefazolin | No | No | No | No |
| Cephalexin | No | No | No | No |
| Monohydrate | | | | |
| Cefaclor | No | No | No | No |
| Loracarbef | No | No | No | No |
| Other Classes of | | | | |
| Oral Agents | | | | |
| Erythromycin | — | — | — | — |
| Estolate | | | | |
| Dirithromycin | No | No | No | No |
| Cinoxacin | No | No | No | No |
| Injectable | | | | |
| agents | | | | |
| Vancmycin HCl | Yes* | Yes** | No | No |
| Tobramycin | No | No | Yes | Yes |
| Cefamandole | No | No | No | No |
| Cefuroxime | No | No | No | No |
| Daptomycin | Yes* | Yes** | No | No |
| Oritavancin | Yes* | Yes** | No | No |

*may require oral dosing for effective treatment of infected warm-blooded animal
**may require oral dosing for internal infection of a warm-blooded animal, and injectable dosing for external infection

We claim:

1. A method of treating a disease in a warm-blooded animal, which disease is attributable to a bio-hazardous bacterial agent, comprising: administering to the animal an antibacterially effective amount of an antibiotic selected from the group consisting of Cephalothin, Cefazolin, Cephalexin monohydrate, Cephalexin HCl, Cefaclor, Loracarbef, Erythromycin estolate, Dirithromycin, Cinoxacin, Vancomycin HCl, Tobramycin, Cefamandole, Cefuroxime, Daptomycin, and Oritavancin.

2. The method claim 1, wherein the bio-hazardous bacterial agent is Bacillus anthracis, Yersinia Pestis, Francisella tularensis, Clostridium botulinin, Clostridium, Perfringens, Brucella abortis, B milletensis, B suis, or Burkholderia mallei.

3. The method of claim 2, wherein the bio-hazardous bacterial agent is Bacillus anthracis, Yersinia Pestis, or Francisella tularensis.

4. The method of claim 3, wherein the bio-hazardous bacterial agent is Bacillus anthracis.

5. The method of any one of claims 1-4, wherein the antibiotic is Cephalexin monohydrate, Cefaclor, or Cefamandole.
